(19) United States
(12) Patent Application Publication
(10) Pub. No.: US 2021/0233661 A1
(43) Pub. Date: Jul. 29, 2021

(54) SYSTEM AND METHOD FOR CREATING GUIDELINE-BASED SUMMARIES FOR IMAGING EXAMS

(71) Applicant: KONINKLIJKE PHILIPS N.V., EINDHOVEN (NL)

(72) Inventors: Lucas de Melo OLIVEIRA, WILMINGTON, MA (US); Qianxi LI, CAMBRIDGE, MA (US)

(21) Appl. No.: 17/267,971

(22) PCT Filed: Aug. 22, 2019
(86) PCT No.: PCT/EP2019/072435

§ 371 Date: Feb. 11, 2021

Related U.S. Application Data
(60) Provisional application No. 62/725,515, filed on Aug. 31, 2018.

(51) Int. Cl. G16H 50/20; G16H 10/60; G16H 40/20; G16H 50/70

(52) U.S. Cl. CPC G16H 50/20; G16H 50/70; G16H 40/20; G16H 10/60

(57) ABSTRACT

A system and method creates guideline-based summaries for imaging exams. The method includes extracting clinical concepts from a patient medical record to create a patient profile. The method includes identifying an appropriate use criteria based on a current clinical workflow. The method includes generating an appropriate use criteria summary including key elements from the patient profile which are used to determine a recommendation based on the appropriate use criteria. The method includes determining whether a user selected action is in violation of the recommendation.

### BACKGROUND

Appropriate Use Criteria (AUC) provide clinical guidelines that specify when it is appropriate to perform a medical procedure or service. An appropriate procedure is one in which the expected health benefits exceed the expected health risks by a wide margin. For a given imaging exam, for example, the AUC may label the exam as “appropriate care”, “may be appropriate care”, or “rarely appropriate care” based on patient information and/or published clinical evidence.

To reduce the cost of advanced diagnostic imaging tests, a requirement for the use of clinical decision support (CDS) was including in the Protecting Access to Medicare Act of 2014. As of Jan. 1, 2017, physicians and other healthcare professionals who order an advanced diagnostic imaging exam must consult with AUC using a qualified CDS system. The ordering physician's consultation of AUC must be documented for the medical facility to receive payment for the imaging exam. The Protecting Access to Medicare Act also directs the Centers for Medicare and Medicaid (CMS) to require prior authorization beginning in 2020 for ordering outlier professionals.

Although AUC have been broadly accepted by clinicians and clinical organizations, these guidelines are not intended to diminish the acknowledged complexity or uncertainty of clinical decision-making and could not be used as a substitute for sound clinical judgment. AUC alone cannot be used to judge treatment appropriateness, as individual patient considerations and physician judgment should take precedence over rigid published AUCs. In addition, documenting evidence associated with AUC has added enormous overhead in a busy clinical workflow. In order to avoid problems with delayed, non-paid or partial reimbursements, medical facilities are required to: (i) identify which AUC should be used for a given episode of care, (ii) search for AUC-specific patient information spread across different medical systems, and (iii) document the information necessary to support their decision. This process can be workflow disruptive and time-consuming.

### SUMMARY

The exemplary embodiments are directed to a method, comprising: extracting clinical concepts from a patient medical record to create a patient profile; identifying an appropriate use criteria based on a current clinical workflow; generating an appropriate use criteria summary including key elements from the patient profile which are used to determine a recommendation based on the appropriate use criteria; and determining whether a user selected action is in violation of the recommendation.

The exemplary embodiment are directed to a system, comprising: a non-transitory computer readable storage medium storing an executable program; and a processor executing the executable program to cause the processor to: extract clinical concepts from a patient medical record to create a patient profile; identify an appropriate use criteria based on a current clinical workflow; generate an appropriate use criteria summary including key elements from the patient profile which are used to determine a recommendation based on the appropriate use criteria; and determine whether a user selected action is in violation of the recommendation.

The exemplary embodiments are directed to a non-transitory computer-readable storage medium including a set of instructions executable by a processor, the set of instructions, when executed by the processor, causing the processor to perform operations, comprising: extracting clinical concepts from a patient medical record to create a patient profile; identifying an appropriate use criteria based on a current clinical workflow; generating an appropriate use criteria summary including key elements from the patient profile which are used to determine a recommendation based on the appropriate use criteria; and determining whether a user selected action is in violation of the recommendation.

### BRIEF DESCRIPTION OF THE DRAWINGS

FIG. 1 shows a schematic drawing of a system according to an exemplary embodiment.

FIG. 2 shows another schematic drawing of the system of FIG. 1.

FIG. 3 shows a flow diagram of a method according to an exemplary embodiment.

FIG. 4 shows a diagram showing a stepwise approach to perioperative cardiac assessment.

### DETAILED DESCRIPTION

The exemplary embodiments may be further understood with reference to the following description and the appended drawings, wherein like elements are referred to with the same reference numerals. The exemplary embodiments relate to systems and methods for applying an AUC for a specific episode of care for a patient and providing an AUC-based summary including key elements for justifying an ordered procedure for the patient. Providing an AUC-based summary may improve physician adherence to guidelines. Most guidelines only provide rigid rules with respect to correct or incorrect courses of action. Providing evidence of the behavior of an AUC may help educate physicians regarding the guidelines. In addition, in some cases, medical professionals are required to gather specific evidence to justify overturning an AUC recommendation. Searching for and organizing the evidence to support the overturning of the AUC recommendation can be time-consuming and workflow disruptive since medical data may be scattered in different medical systems and the evidence required is linked o a specific patient profile and AUC. The systems and methods of the present disclosure automatically identify an AUC based on a clinical workflow, detect if an AUC has been violated, and then automatically extract and organize AUC-based evidence that may be used to support a physician's decision—e.g., an ordered procedure in violation of the AUC. Thus, the exemplary systems and methods streamline the clinical workflow and provide clinical decision support. Although the exemplary embodiments specifically describe the application of AUCs in an echocardiogram workflow, it will be understood by those of skill in the art that systems and methods of the present disclosure may utilize any of a variety of clinical guidelines within any of a variety of medical domains such as, for example, radiology and pathology.

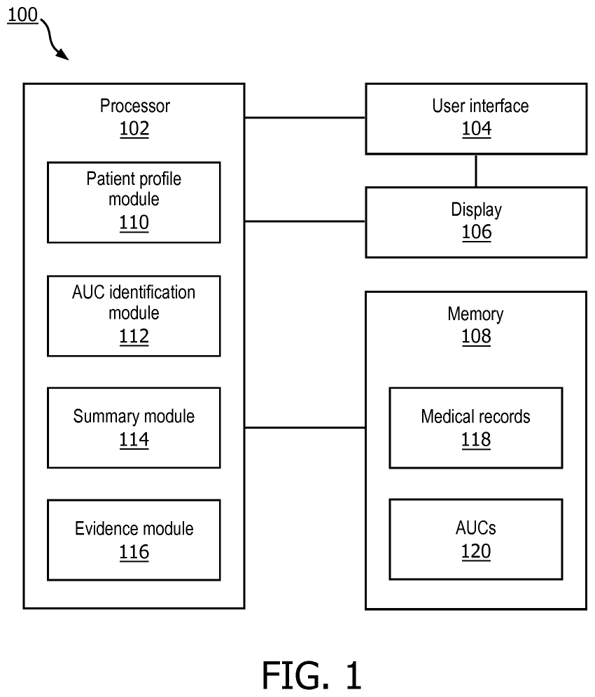

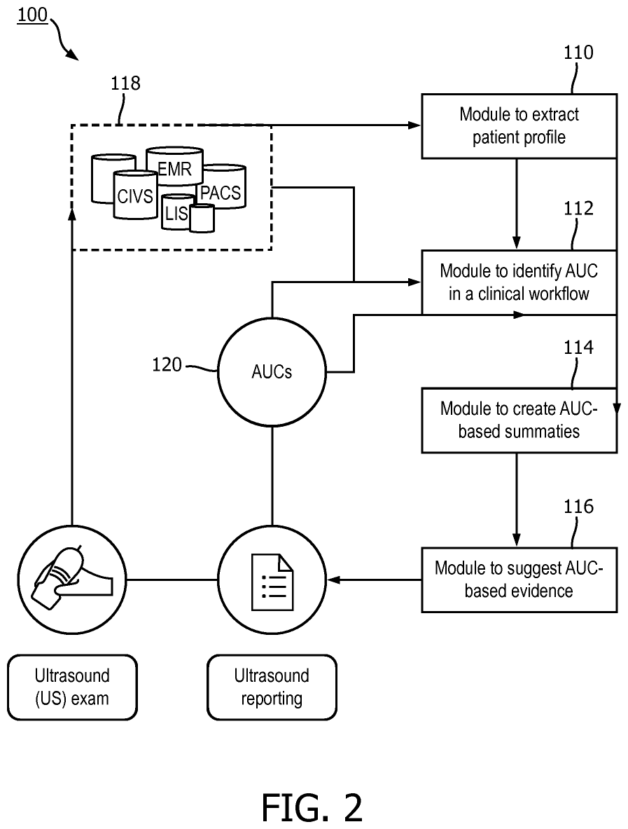

As shown in FIGS. 1 and 2, a system 100, according to an exemplary embodiment of the present disclosure, applies an AUC for a specific episode of care and creates a summary based on key elements for the applied AUC. The system 100, as shown in FIG. 1, comprises a processor 102, a user interface 104, a display 106, and a memory 108. The memory 108 includes a database of patient medical records 118 along with a database of AUCs 120 that may be applied for the specific episode of care. The medical records may include, for example, medical notes, medication lists, and/or laboratory notes from various medical sources (e.g., EMR, PACS, CIVS, LIS, etc.). It is noted that memory 108, including medical records 118 and AUCs 120 shown in system 100, may be distributed among many different storage devices. In fact, with the advent of Electronic Medical Records (EMRs), a patient may have separate electronic records with different doctors, hospitals, care organizations, insurance companies, etc. Thus, one of the issues associated with applying an AUC for a specific episode of care is to ensure that all relevant medical records are used.

The processor 102 may include a patient profile module 110, a AUC identification module 112, a summary module 114 and an evidence module 116. As shown in FIG. 2, the patient profile module extracts data from a variety of data sources such as, for example, EMR, CIVS, PACS, and LIS included in the patient's medical record 118 to create a patient profile The AUC identification module 112 identifies an AUC from a database of potential AUCs 120 based on a clinical workflow, which may be identified from an imaging exam being reviewed (e.g., ultrasound exam) and/or credentials of a user (e.g., radiologist, cardiologist). The summary module 114 generates a summary including key elements, identified from the patient profile created by the patient profile module 110, which may be used to determine whether potential options of the AUC identified by the AUC identification module 112 are appropriate, maybe appropriate or rarely appropriate. Upon determining whether an option and/or ordered procedure is appropriate, the evidence module 116 may generate an evidence summary including key elements supporting a user's violation of an AUC recommendation (e.g., appropriate, maybe appropriate, rarely appropriate). Those skilled in the art will understand that the modules 110-116 may be implemented by the processor 102 as, for example, lines of code that are executed by the processor 102, as firmware executed by the processor 102, etc.

A user (e.g., physician) may input user selections on the user interface 104 to, for example, initiate or trigger an AUC application, order an imaging exam or other procedure, and/or edit or set parameters for the modules 110-116. For example, the AUC application and/or AUC-based summary generation may be triggered when ordering or planning for patient treatment. Thus, for example, when the user orders a test via the user interface 104, the application may be triggered automatically. In another example, the user may manually select to initiate the AUC application/AUC-based summery to check for compliance and/or assist in generating evidence for a treatment plan. Evidence compiled by the AUC-based summary can provide justification when filing for a reimbursement. Patient profiles and/or AUC based summaries may be displayed to the user on the display 106.

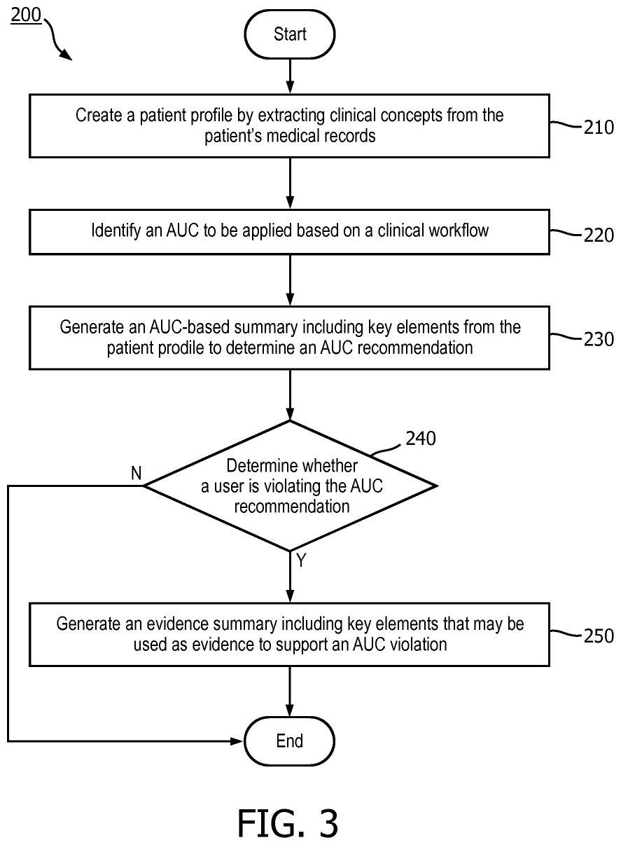

FIG. 3 shows an exemplary method 200 for generating an AUC-based summary to support clinical decision-making and/or provide documentation to support clinical actions and/or orders in violation of AUC. The AUC-based summary includes key elements for determining whether an action is appropriate under the AUC guidelines. A user such as a physician or other medical professional may utilize the summary to determine whether to adhere to the AUC recommendation or to override the recommendation. The method 200 may be triggered or initiated by an event or user. In one example, the method 200 may be triggered by the reading or analysis of an acquired image. In another example, the method 200 may be triggered when the user orders a procedure for the patient. It will be understood by those of skill in the art, however, that the method 200 may be initiated in response to any of a variety of triggers which may be selected and/or input by the user interface 104.

In 210, the patient profile module 110 creates a patient profile for a particular patient by extracting clinical concepts from a patient's medical record. Clinical concepts may include the patient's clinical presentations (e.g., left ventricle is enlarged, moderated tricuspid regurgitation, etc.), which may be relevant for determining an appropriate action. Clinical concepts may be extracted from a variety of different medical data sources such as, for example, medical notes, medication lists and laboratory reports. Medical records may be accessed using, for example, SOA services or HL7. The clinical concepts may be extracted using any of a variety of statistical, Natural Language Processing (NLP), or machine learning approaches. For example, admission notes may be an important source of information for the patient profile, indicating patient history and reason for admission. Admission notes are generally in free-text format so that the patient profile module 110 may utilize NLP to extract clinical concepts from the natural language.

In 220, the AUC identification module 112 selects an AUC for a given patient based on a clinical workflow. The clinical workflow may be determined in any of a variety of ways. In one embodiment, the clinical workflow may be identified based on a given medical event. For example, the clinical workflow may be determined by identifying the user involved in the treatment of the patient (e.g., radiologist, interventional cardiologist, anesthesiologist) or by the electronic system being utilized by the user (e.g., Radiology Information System (RIS), Cardiovascular Information System (CVIS)). In another embodiment, the clinical workflow may be identified by study schedules and/or information associated with a procedure. In the cardiology domain, for example, the clinical workflow may identify the coronary revascularization AUCs or the echocardiogram AUCs. Once the clinical workflow has been identified, the AUC identification module 112 provides a more selective set of AUC based on the patient's medical record. For example, if the clinical workflow is identified as “cardiovascular revascularization workflow”, the AUC identification module 112 may further identify a patient diagnosis (e.g., stable ischemic heart disease, acute coronary syndrome) by inspecting, for example, patient symptoms, exams performed, labs ordered and/or medications prescribed.

In another embodiment, machine learning may be utilized to select AUCs for a given input. The AUC identification module 112 may be trained using statistical methods. For example, a training data set may include patient diagnoses, medication lists, lab results, previous exam, healthcare professionals involved and the associated AUCs to create a model which assigns AUCs based on the given inputs.

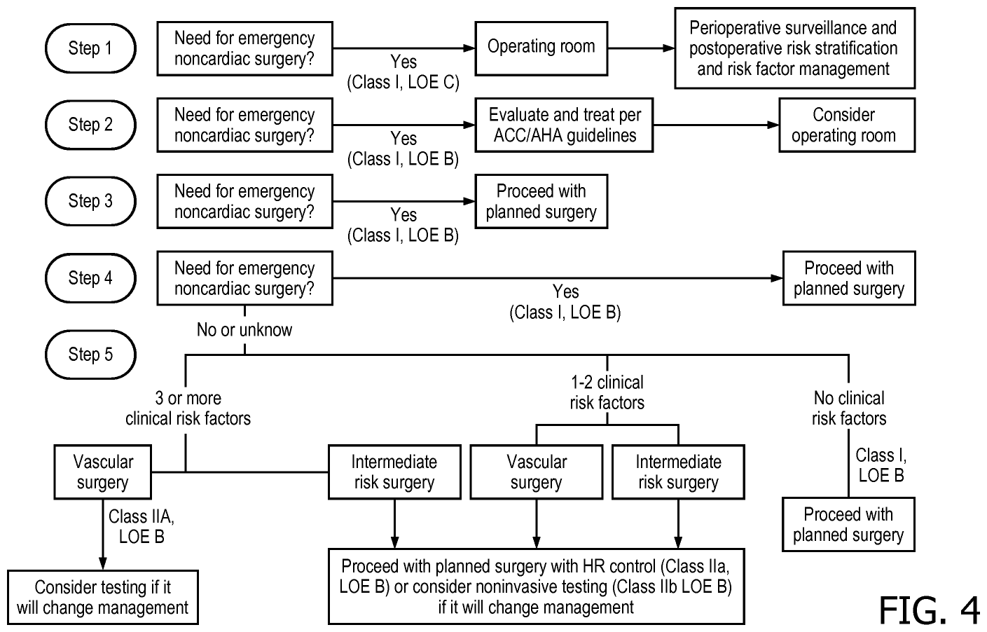

In yet another embodiment, a decision tree approach may be utilized to identify an AUC associated with a specific procedure. Decision tree logic may be published by scientific societies such as, for example, the Journal of the American College of Cardiology, and may include appropriate treatment options based on a number of conditions. For example, FIG. 4 shows a decision tree for approaching perioperative cardiac assessment. Potential treatment options include, for example, “Consider Operating Room” and “Proceed with Planned Surgery”. If a procedure is planned or ordered for a patient, the associated AUC may be identified by comparing the ordered procedure with the “planned surgery” of the decision tree. For example, if a Coronary Artery Bypass Grafting (CABG) is ordered for a patient, CABG is compared with the “planned surgery” treatment option of various decision trees to identify the specific associated AUC decision tree. NLP may be utilized to compare the ordered procedure with the planned procedure of the decision tree.

In 230, the summary module 114 creates an AUC-based summary based on key elements for a given AUC. Key elements may include conditions or factors used to determine whether an action is appropriate, may be appropriate or rarely appropriate. For example, key elements for a perioperative cardiac assessment may include factors for determining whether there is a need for emergency noncardiac surgery, whether there is an active cardiac condition, whether the planned surgery is a low risk surgery, whether the functional capacity is greater than or equal to 4 METs without symptoms, etc. Key elements for coronary revascularization may include clinical presentation (e.g., acute coronary syndrome, stable angina), severity of angina (e.g., asymptomatic, Canadian Cardiovascular Society (CCS) Class I, II, III or IV), extent of ischemia on non-invasive testing and the presence of congestive heart failure.

The AUC-based summary is created using the patient profile created in 210 and the AUC selected in 220. The AUC-based summary may be displayed to the user on the display 106. In one embodiment, items in the summary may be organized based on frequency of the key elements and/or based on the AUC flow of, for example, the AUC decision tree. In another embodiment, the key elements may be organized using a predetermined order such as, for example, chronological, clinical relevance and/or anatomy. The organized list of key elements may also be filtered via anatomical structure, exam modality or other predetermined filters.

In yet another embodiment, the summary may be created using a set of pre-defined templates created by healthcare professionals. The template may be used to generate a standardized presentation of key elements. User interaction with the system 100 (e.g., edits to the summary) may be used to train the summary module 114. Thus, templates may be generated by combining the AUC decision tree and a machine learning approach. In this embodiment, the summary module 112 may be trained using a finite set of labeled or unlabeled input such as, for example, previous summaries, AUC key elements and patient profile features. These features may be used to train a model that can automatically output an AUC-based summary.

According to an example using “Appropriate Use Criteria for Coronary Revascularization in Patients with Stable Ischemic Heart Disease,” a PCI (Percutaneous Coronary Intervention) is rarely appropriate for a low-risk patient with one-vessel disease findings on non-invasive tests such as an echocardiogram or EKG. The summary module 114 will create a summary showing some key elements that can help clinicians understand the AUC recommendation and evidence that may be used to support their decision. Key elements may include, for example, evidence to show that a patient is a low-risk patient (e.g., no new ST segment changes or exercise-induced chest pain symptoms, normal stress or no change of limited resting wall motion abnormalities during stress, and no coronary stenosis>50% on CCTA), number of vessels with stenosis, patient history, list of previous diagnostic exam, and medication lists. The key elements extracted from the patient profile may be used to generate the AUC-based summary for the patient. If, for example, a patient has a clinical element such as a family history of sudden cardiac death in his/her family or hypertrophic cardiomyopathy, this information may be used to override the AUC recommendation and proceed with the PCI order.

Based on the key elements included in the AUC-based summary, a user may decide to follow the AUC recommendation or override the AUC recommendation. In 240, the evidence module 116 determines whether there is a potential AUC violation—e.g., the user does not follow the AUC violation. If the user decides to follow the AUC recommendation, no further action may be taken. For example, if the user ordered a procedure and this procedure is determined to align with the AUC recommendation, the method 200 ends. In another example, if, based on the identified AUC and the patient profile, the AUC recommends a particular action and the user follows this recommended action, the method 200 ends. If, however, the user overrides the AUC recommendation, the method 200 proceeds to 250. It will be understood by those of skill in the art that this determination may be made based upon user input in response to the AUC recommendation and/or a previously ordered procedure, which triggered the method 200.

The evidence module 116 may implement a machine learning and/or rule-based AUC violation detection approach using the AUC definitions. Using the AUC for Coronary Revascularization in Patients with Stable Ischemic Heart Disease as an example, the rule-based AUC violation detection approach for identifying an AUC violation may be:
If (“patient is low risk” and “there is one vessel stenosis”)
Then PCI is “rarely appropriate”.

In 250, when it has been determined that the user is in violation of the AUC recommendation, the evidence module 116 generates an evidence summary including key elements which may be used to support the AUC violation. Using the above example of the PCI order, the evidence summary may include key elements such as, for example, family history of sudden cardiac death in his/her family or hypertrophic cardiomyopathy. This evidence summary may be displayed to the user on the display 106 and/or may be stored to the memory 108 to provide documentation supporting the user determination.

Those skilled in the art will understand that the above-described exemplary embodiments may be implemented in any number of manners, including, as a separate software module, as a combination of hardware and software, etc. For example, the patient profile module 110, the AUC identification module 112, the summary module 114, and the evidence module 116 may be programs containing lines of code that, when compiled, may be executed on the processor 102.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

What is claimed is:

1. A method, comprising:
extracting clinical concepts from a patient medical record to create a patient profile;
identifying an appropriate use criteria based on a current clinical workflow;
generating an appropriate use criteria summary including key elements from the patient profile which are used to determine a recommendation based on the appropriate use criteria; and
determining whether a user selected action is in violation of the recommendation.

2. The method of claim 1, further comprising, when there is a violation of the recommendation, generating an evidence summary including key elements that support the violation of the recommendation.

3. The method of claim 2, storing the evidence summary in a memory.

4. The method of claim 1, wherein the clinical concepts are extracted via one of a statistical approach, a natural language processing, and a machine learning.

5. The method of claim 1, wherein identifying the appropriate use criteria includes identifying the current clinical workflow based on one of credentials of the user, an electronic system being utilized by the user, a study schedule, or an ordered procedure.

6. The method of claim 1, wherein identifying the appropriate use criteria includes selecting the appropriate use criteria from a database of potential appropriate use criteria.

7. The method of claim 1, wherein the appropriate use criteria summary is organized based on one of a frequency of key elements, a decision-based flow diagram, a chronological order, clinical relevance and anatomy.

8. The method of claim 1, further comprising filtering the key elements of the appropriate use criteria based on one of anatomical structure and exam modality.

9. The method of claim 1, wherein the appropriate use criteria summary is generated using a pre-defined template stored in a memory.

10. The method of claim 1, further comprising receiving user inputs for editing the appropriate use criteria summary.

11. A system, comprising:
a non-transitory computer readable storage medium storing an executable program; and
a processor executing the executable program to cause the processor to:
extract clinical concepts from a patient medical record to create a patient profile;
identify an appropriate use criteria based on a current clinical workflow;
generate an appropriate use criteria summary including key elements from the patient profile which are used to determine a recommendation based on the appropriate use criteria; and
determine whether a user selected action is in violation of the recommendation.

12. The system of claim 10, wherein the processor executes the executable program to cause the processor to:
generate, when it is determined that there is a violation of the recommendation, an evidence summary including key elements that support the violation of the recommendation.

13. The system of claim 12, wherein the processor executes the executable program to cause the processor to:
store the evidence summary in a memory.

14. The system of claim 11, wherein the processor executes the executable program to cause the processor to:
extract the clinical concepts via one of a statistical approach, a natural language processing, and a machine learning.

15. The system of claim 11, wherein the processor executes the executable program to cause the processor to:
identify the appropriate use criteria by identifying the current clinical workflow based on one of credentials of the user, an electronic system being utilized by the user, a study schedule, or an ordered procedure.

16. The system of claim 11, wherein the processor executes the executable program to cause the processor to:
identifying the appropriate use criteria by selecting the appropriate use criteria from a database of potential appropriate use criteria.

17. The system of claim 11, wherein the processor executes the executable program to cause the processor to:
organize the appropriate use criteria summary is based on one of a frequency of key elements, a decision-based flow diagram, a chronological order, clinical relevance and anatomy.

18. The system of claim 11, wherein the processor executes the executable program to cause the processor to:
filter the key elements of the appropriate use criteria based on one of anatomical structure and exam modality.

19. The system of claim 11, wherein the processor executes the executable program to cause the processor to:
generate the appropriate use criteria summary using a pre-defined template stored in a memory.

20. The system of claim 11, wherein the processor executes the executable program to cause the processor to:
receive user inputs for editing the appropriate use criteria summary.

21. A non-transitory computer-readable storage medium including a set of instructions executable by a processor, the set of instructions, when executed by the processor, causing the processor to perform operations, comprising:
extracting clinical concepts from a patient medical record to create a patient profile;
identifying an appropriate use criteria based on a current clinical workflow;
generating an appropriate use criteria summary including key elements from the patient profile which are used to determine a recommendation based on the appropriate use criteria; and
determining whether a user selected action is in violation of the recommendation.
